(19) United States
(12) Patent Application Publication
(10) Pub. No.: US 2021/0228661 A1
(43) Pub. Date: Jul. 29, 2021

(54) ANTI-PARASITIC COMPOSITION

(71) Applicant: FermentationExperts A/S, Bække (DK)

(72) Inventors: Jens Høffner Legarth, Bække (DK); Søren Kjærulff, Holte (DK)

(21) Appl. No.: 15/734,931

(22) PCT Filed: Jun. 7, 2019
(86) PCT No.: PCT/EP2019/065015

§ 371 Date: Dec. 3, 2020

(30) Foreign Application Priority Data
Jun. 13, 2018 (DK) PA 2018 00269
Apr. 6, 2019 (DK) PA 2019 00426

(51) Int. Cl. A61K 36/03; A23K 50/80; A23K 20/00; A61K 9/00; A61K 9/16; A61P 33/14

(52) U.S. Cl. CPC A61K 36/03; A23K 50/80; A23K 20/00; A61P 33/14; A61K 9/009; A61K 9/16; A61K 9/0053

(57) ABSTRACT

An anti-parasitic composition is provided that includes at least one seaweed material, at least one plant material, or a combination of at least one seaweed material and at least one plant material, for inhibiting, reducing, and/or suppressing growth of at least one parasite.

### TECHNICAL FIELD OF THE INVENTION

The present invention relates to an anti-parasite composition. In particular, the present invention relates to an anti-parasitic composition suitable for inhibiting, reducing and/or suppressing growth of at least one parasite and a feed product comprising the anti-parasite composition.

### BACKGROUND OF THE INVENTION

Parasites are part of a large group of organisms belonging to the group of eukaryotes. Parasites are different from bacteria and viruses. The key difference between parasites and bacteria is that parasites have an organized nucleus, whereas, bacteria are unicellular microscopic prokaryotes organisms having just a single stranded DNA. In addition, parasites contain endoplasmic reticulum, Golgi apparatus, lysosomes, mitochondria, micro-filaments, microtubules, and centrosomes.

Parasites is an organism that lives and feeds on or in an organism of a different species (host). While living in association, the parasite gets benefits from the host harming or causing infections to the host body.

Parasitic infections can usually be treated with anti-parasitic drugs. Albendazole and mebendazole are two anti-parasitic drugs used to treat the hookworm nematode that parasites on the intestine of mammals. The drugs are generally administered to entire herds of pigs in intensive farming and results in a very costly option. Furthermore, piglets and grown up pigs become re-infected within a few months after deparasitation and the treatment has to repeatedly be administered and drug resistance may occur.

Another medication administered to kill parasitic infections, such as worm infections, has been the drug pyrantel pamoate. However, pyrantel pamoate has the side effects include nausea, headache, dizziness, trouble sleeping, and rash

For other parasitic infections, there is no treatment, and, in the case of serious symptoms, medication intended to kill the parasite is administered, whereas, in other cases, symptom relief options are used. Recent papers have also proposed the use of viruses to treat infections caused by protozoa.

Thus, in cases where treatment of the parasitic infection is possible, it may be difficult, expensive, involve various side effects, not sufficient and reoccurring treatment may be required, and there is a risk of resistance to the medical treatments.

One of the parasites offered most attention is Lepeophtheirus salmonis, or salmon louse, which is a very troublesome parasite, and has resulting in huge investments and costs to find proper solutions and treatments in the benefit for the billion-dollar businesses of fish farmers.

Lepeophtheirus salmonis/salmon louse is a natural parasite of saltwater salmon belonging to the subclass copepod. The lice are eating skin, mucus and blood of the salmon and can create large wounds and harm the quality of the salmon.

Combating Lepeophtheirus salmonis/salmon louse is traditionally done with drugs, sucker fish or by mechanical methods.

The drugs are either added to the feed or given as a bath. Bathing can be done in cages covered with full tarpaulin, or in a wellboat. If there is a resistant loop, repeated treatments with the same active substance will select these to form an increasing portion of the loop population, with the accompanying poor treatment effect.

Drugs are and have been the most common way to fight salmon lice. This is done either by adding the medication to the feed or by bathing. When bathing, the cage is enclosed by a tight tarpaulin. The bathing can also be done on board a wellboat. Loose larvae from resistant salmon lice will spread between aquaculture plants and infections may escalate. In Norway, there is now widespread resistance to most of the approved drugs against salmon lice.

Due to the increasing resistance, various non-medical measures have been taken. The most common of non-medical treatment is the use of clean fish/sucker fish that eats the salmon lice. The use of clean fish/sucker fish is most effective on less salmon and when it is not too cold in the water.

Another non-medical treatment is a mechanical treatment which has become more and more common in recent years due to the fact that the salmon lice has developed resistance to the drugs and the problems of using clean fish/sucker fish. The mechanical treatment may face the result in problems with brushing the salmon resulting in lesser fish quality and increased costs as large amounts of treatment water is used.

Hence, an improved anti-parasitic composition for controlling (inhibiting, reducing and/or suppressing growth of at least one parasite) would be advantageous, and in particular a more efficient and/or reliable anti-parasitic composition, which is inexpensive, reduce or even avoid various side effects, reduce or even avoid repeated reoccurring treatments, and reduce or avoid the risk of resistance to anti-parasitic composition, would be advantageous.

### SUMMARY OF THE INVENTION

Thus, an object of the present invention relates to an improved anti-parasitic composition for controlling, e.g. inhibiting, reducing and/or suppressing, growth of at least one parasite.

In particular, it is an object of the present invention to provide an anti-parasitic composition that solves the above-mentioned problems of the prior art with lack of efficiency and/or reliability, which anti-parasitic composition, is inexpensive, reduce or even avoid various side effects, reduce or even avoid repeated reoccurring treatments, and reduce or avoid the risk of resistance to anti-parasitic composition.

Thus, one aspect of the invention relates to an anti-parasitic composition comprising at least one seaweed material, at least one plant material or a combination of at least one seaweed material and at least one plant material for inhibiting, reducing and/or suppressing growth of at least one parasite.

Another aspect of the present invention relates to a feed product comprising the anti-parasite composition according to the present invention.

Yet, another aspect of the present invention relates to the use of the anti-parasite composition according to the present invention for the treatment, prophylaxis and alleviation of a parasitic infection.

Still another aspect of the present invention relates to the use of the anti-parasite composition according to the present invention for inhibiting, reducing and/or suppressing growth of at least one parasite in a food product and/or in a feed product.

### DETAILED DESCRIPTION OF THE INVENTION

Treatment of parasitic infections have shown to be much more complicated compared to e.g. bacterial infections or viral infections as the parasites has stronger survivability and may often require repeated reoccurring treatments. Without being bound by theory, it is expected that this more complicated treatment may be caused by e.g. the different life stages in particular eukaryotic parasites go through (including stages where the parasites are dormant or in a latent form), the presence of the cell wall and an organized nucleus providing an independent organism comprising endoplasmic reticulum, Golgi apparatus, lysosomes, mitochondria, micro-filaments, microtubules, and centrosomes.

The inventors of the present invention surprising found a composition that showed to enter the parasite cell, influence the nucleus, the endoplasmic reticulum, the Golgi apparatus, the lysosomes, the mitochondria, the micro-filaments, the microtubules, and/or the centrosomes resulting in a control, e.g. inhibiting, reducing and/or suppressing, of the growth of at least one parasite.

Thus, a preferred embodiment of the present invention relates to an anti-parasitic composition comprising at least one seaweed material, at least one plant material or a combination of at least one seaweed material and at least one plant material for inhibiting, reducing and/or suppressing growth of at least one parasite.

In the context of the present invention, the term “anti-parasitic composition” relates to a composition according to the present invention for the treatment of parasitic infections, preferably by destroying the parasite or inhibiting, reducing and/or suppressing parasitic growth.

In another preferred embodiment of the present invention wherein the anti-parasitic composition is consisting essentially of at least one seaweed material, at least one plant material or a combination of at least one seaweed material and at least one plant material for treating, alleviating and/or preventing growth of at least one parasite.

In the context of the present invention, the term “comprising”, which may be synonymous with the terms “including”, “containing” or “characterized by”, relates to an inclusive or open-ended listing of features and does not exclude additional, unrecited features or method steps. The term “comprising” leaves the claim open for the inclusion of unspecified ingredients even in major amounts.

In the context of the present invention, the term “consisting essentially of”, relates to a limitation of the scope of a claim to the specified features or steps and those features or steps, not mentioned and that do not materially affect the basic and novel characteristic(s) of the claimed invention.

In an embodiment of the present invention, the anti-parasitic composition according to the present invention may be used as an ingredient in a feed product or in a food product, resulting in a control of the parasitic growth, e.g. inhibiting, reducing and/or suppressing the parasitic growth or even killing the parasites present in the feed product or in the food product.

In another embodiment of the present invention, the anti-parasitic composition may be used for human or animal administration. When the anti-parasitic composition is used for human or animal administration it may be used as a product as such, or it may be used as an ingredient added to the feed product or food product. When the anti-parasitic composition is used for human or animal administration, controlling, e.g. inhibition, reduction and/or suppression, of the parasitic grow may result in a treatment, an alleviation and/or a prevention of the growth of at least one parasite.

The at least one seaweed material forming part of the anti-parasitic composition may preferably comprises at least one fermented seaweed material. Furthermore the at least one seaweed material is a fermented seaweed material.

The term “at least one seaweed material” implies that different seaweed materials may be used. In an embodiment of the present invention the anti-parasitic composition comprises at least one seaweed material, such as at least two seaweed materials, e.g. at least three seaweed materials, such as at least four seaweed materials.

When the anti-parasitic composition comprises two or more seaweed materials, the seaweed materials may be of different origin and/or the seaweed materials may be of same origin but one being fermented and another non-fermented.

In an embodiment of the present invention wherein the seaweed material may be a unicellular alga or a multicellular macroalgae.

In a further embodiment of the present invention the multicellular macroalgae may be selected from brown macroalgae, red macroalgae, and/or green macroalgae.

In another embodiment of the present invention the brown macroalgae may be selected from one or more of kelps, Saccharina latissimi (Laminaria saccharina), Laminaria digitate, Ascophyllum nodosum, Laminaria hyperborean, or a mixture hereof.

The at least one plant material forming part of the anti-parasitic composition may preferably comprises at least one fermented plant material. Furthermore, the at least one plant material may be a fermented plant material.

In the present context, the term “plant material” relates to capable of performing photosynthesis.

The term “at least one plant material” implies that different plant materials may be used. In an embodiment of the present invention the anti-parasitic composition comprises at least one plant material, such as at least two plant materials, e.g. at least three plant materials, such as at least four plant materials.

When the anti-parasitic composition comprises two or more plant materials, the plant materials may be of different origin and/or the plant materials may be of same origin but one being fermented and another being non-fermented.

In an embodiment of the present invention the plant material may be selected from at least one proteinaceous plant material. The proteinaceous plant material may be selected from eudicot plants, angiosperm plants, and/or rosid plants.

Preferably the proteinaceous plant material may be selected from Brassicale plants.

In an embodiment of the present invention the Brassicale plants is selected from the Brassicaceae family or the Cruciferae family.

In a further embodiment of the present invention the Brassicaceae family or the Cruciferae family may be selected from at least one of a Brassica genus; sun flower; palm; soya, field beans, Lupins; or a combination hereof. Preferably, at least one Brassica genus may be selected from one or more species such as Brassica napus; Brassica oleracea; Brassica campestris; Brassica nigra; Sinapis alba (Brassica alba); Brassica juncea; Brassica rapa or mixtures hereof.

In yet an embodiment of the present invention the at least one Brassica genus may be selected from the group consisting of: including rape, rapeseed, canola, cabbage, broccoli, cauliflower, kale, Brussels sprouts, collard greens, savoy, kohlrabi, gai lan, white mustard, Indian mustard, Chinese mustard, and black mustard seed powder.

The anti-parasitic composition may comprise a combination of at least one seaweed material as defined above, and at least one plant material as defined above.

Preferably, the combination of at least one seaweed material as defined above, and at least one plant material as defined above may be a combination of at least one fermented seaweed material and at least one fermented plant material.

In the present context the term “fermented” relates to a material (a plant material, a seaweed material or a combination of the plant material and the seaweed material) relates to a controlled metabolic process of the material by adding a predetermined amount of fermenting microorganisms to the material allowing the microorganism and the material to interact breaking down the material.

The fermenting microorganisms may be a health-enhancing microorganism.

Hence, in an embodiment of the present invention the anti-parasitic composition may comprise one or more health-enhancing microorganism, preferably the health-enhancing microorganism may be a health-enhancing yeast and/or a health-enhancing bacterium, even more preferably the health-enhancing microorganism may be a health-enhancing bacterium.

The health-enhancing bacteria may comprise one or more probiotics. The one or more probiotics and/or the one or more health-enhancing microorganism may comprise at least one lactic acid bacterial strain.

In the context of the present invention, the term “probiotic” relates to live microorganisms that when administered in adequate amounts, confer a health benefit on the host.

In a preferred embodiment of the present invention the at least one lactic acid bacterial strain may be selected from the group consisting of the genus Enterococcus, Lactobacillus, Pediococcus, Lactococcus, or Bifidobacterium or combinations thereof.

In a further embodiment of the present invention the one or more lactic acid bacterial stain(s) may be selected from the group consisting of Pediococcus pentosaceus; Pendiococcus acidilactici; Lactobacillus plantarum, Lactobacillus rhamnosus, Enterococcus faecium, Lactobacillus acidophilus, Bifidobacterium lactis, Bifidobacterium longum, Bifidobacterium bifidum, Lactobacillus salivarius, Lactobacillus pentoses, Lactobacillus vaginalis, Lactobacillus xylosus and a combination thereof.

In an embodiment of the present invention the health-enhancing microorganism may be the main microorganism present in the anti-parasitic composition. Preferably, the main microorganism is a lactic acid bacterium. Even more preferably the main microorganism may be selected from the groups consisting of Pediococcus pentosaceus; Pendiococcus acidilactici, Lactobacillus plantarum, Lactobacillus rhamnosus, Enterococcus faecium, Lactobacillus acidophilus, Bifidobacterium lactis, Bifidobacterium longum, Bifidobacterium bifidum, Lactobacillus salivarius, Lactobacillus pentoses, Lactobacillus vaginalis, and Lactobacillus xylosus; preferably, the main lactic acid bacteria present in the composition is Lactobacillus plantarum.

In the context of the present invention the term “main microorganism” relates to the microorganism present in highest amount, determined on a weight/weight ratio.

During fermentation a group of microorganisms may be used for fermenting the plant material to provide a co-fermentation. The co-fermentation may be a mixture of different microorganisms (such as a mixture of yeasts, fungus, and/or bacteria) or a mixture of different bacteria. Preferably the co-fermentation comprises a mixture of different bacterial strains. In an embodiment of the present invention the anti-parasitic composition comprises one or more bacterial strains, e.g. two or more bacterial strains, such as three or more bacterial strains, e.g. four or more bacterial strains, such as 7 or more bacterial strains, e.g. 10 or more bacterial strains, such as 15 or more bacterial strains, e.g. 20 or more bacterial strains, such as 25 or more bacterial strains, e.g. 30 or more bacterial strains, such as 35 or more bacterial strains, e.g. 40 or more bacterial strains. Preferably, the bacterial strains may be one or more lactic acid bacterial strains.

In a further embodiment of the present invention the one or more lactic acid bacteria stain(s) may be selected from the group consisting of one or more of Enterococcus faecium MCIMB 30122, Lactobacillus rhamnosus NCIMB 30121, Pediococcus pentosaceus HTS (LMG P-22549), Pendiococcus acidilactici NCIMB 30086 and/or Lactobacillus plantarum LSI (NCIMB 30083).

In order to provide the desired effects, the anti-parasitic composition should have a high content of viable lactic acid bacteria. In an embodiment of the present invention the anti-parasitic composition comprises one or more lactic acid bacterial strain(s) in a total amount in the range of 10⁵-10¹² CFU per gram of the anti-parasitic composition, such as in the range of 10⁸-10¹² CFU per gram, e.g. in the range of 10⁷-10¹¹ CFU per gram, such as in the range of 10⁸-10¹¹ CFU per gram, e.g. in the range of 10⁸-10¹⁰ CFU per gram.

During fermentation, various metabolites may be formed, which in combination, may assist in the inhibiting, reducing and/or suppressing growth and/or the treatment, prophylaxis and alleviation of a parasitic infection.

As mentioned above, it was surprising to the inventors that the anti-parasitic composition according to the present invention was active in controlling growth of one or more anti-parasitic composition. Preferably, the parasite may be an ectoparasites or an endoparasite.

In the present context, the term “endoparasite” relates to parasites that live inside the host and the term “ectoparasites” relates to parasites that live outside the host.

In an embodiment of the present invention, the endoparasite may be a plant parasite, a parasitic worm, or protozoa.

The parasitic worm may preferably be selected from the group consisting of Cestoda phylum, Nematoda phylum, Acanthocephala phylum and Trematoda phylum.

In an embodiment of the present invention, the ectoparasite may be an Arthropoda phylum, an Annelid phylum, a Monogenean, a Mollusca phylum, a Chordata phylum, Plant parasite. The Arthropoda phylum may preferably be selected from a Crustacea subphylum. Even more preferably, the Crustacea subphylum may be selected from a class Maxillopoda. The class Maxillopoda may preferably be selected from a Copepoda subclass and the Copepoda subclass may preferably be selected from a Siphonostomatoida order. The Siphonostomatoida order may preferably be selected from a Caligidae family.

In a preferred embodiment of the present invention the parasite, and/or the Caligidae family may be selected from a Lepeophtheirus genus. Preferably, the Lepeophtheirus genus is selected from a L. salmonis species.

The salmon louse, Lepeophtheirus salmonis, may occurs in cold temperate waters of the mainly in the northern hemisphere. L. salmonis is an ectoparasite, and even it is called a salmon louse it, may occur on all salmonid species; salmon, trout and char. They may be found on other fish species as a stop-gap measure while seeking out their salmonid host.

The anti-parasitic composition according to the present invention the anti-parasitic composition may comprise a lactic acid concentration of at least 50 mM, such as at least 100 mM, such as 100-1000 mM, such as 100-500 mM, such as 100-300 mM, such as 100-200 mM, such as 150-500 mM, such as 200-500 mM or such as 300-500, mM lactic acid.

In an embodiment of the present invention the anti-parasitic composition may comprise a pH-value below pH 6.5, such as below pH 6.0, e.g. below pH 5.5, such as below pH 5.0, e.g. in the range of pH 3.0-6.5, such as in the range of pH 3.0-6.0, e.g. in the range of pH 3.1-5.5, such as in the range of pH 5.0-3.2, such as in the range 3.3-4-2, such as 3.4-4.0, such as 3.5-3.8, such as 3.7-4.2, such as 3.7-4.0, or such as 3.8-4.2.

The anti-parasitic composition according to the present invention may preferably comprises a fibrous material. Preferably the anti-parasitic composition comprises the fibrous material originating from the plant material and/or the seaweed material.

In an embodiment of the present invention the anti-parasitic composition comprises more than 5 g fibrous material per kg dried anti-parasitic composition, such as more than 10 g fibrous material per kg dried composition, e.g. more than 15 g fibrous material per kg dried composition, such as more than 20 g fibrous material per kg dried composition, e.g. more than 25 g fibrous material per kg dried composition, such as more than 50 g fibrous material per kg dried composition, e.g. more than 75 g fibrous material per kg dried composition, such as more than 100 g fibrous material per kg dried composition, e.g. more than 150 g fibrous material per kg dried composition, such as more than 200 g fibrous material per kg dried composition, e.g. more than 250 g fibrous material per kg dried composition, such as more than 300 g fibrous material per kg dried composition.

In a further embodiment of the present invention the anti-parasitic composition comprises between 5-400 g fibrous material per kg dried anti-parasitic composition, such as between 10-300 g fibrous material per kg dried composition, e.g. between 15-250 g fibrous material per kg dried composition, such as between 20-200 g fibrous material per kg dried composition, e.g. between 25-150 g fibrous material per kg dried composition, such as between 50-125 g fibrous material per kg dried composition, e.g. between 75-100 g fibrous material per kg dried composition.

The anti-parasitic composition according to the present invention may preferably comprises a starch material. Preferably the anti-parasitic composition comprises the starch material originating from the plant material and/or the seaweed material.

In an embodiment of the present invention wherein the composition may comprise more than 5 g starch material per kg dried composition, such as more than 10 g starch material per kg dried composition, e.g. more than 15 g starch material per kg dried composition, such as more than 20 g starch material per kg dried composition, e.g. more than 25 g starch material per kg dried composition, such as more than 50 g starch material per kg dried composition, e.g. more than 75 g starch material per kg dried composition, such as more than 100 g starch material per kg dried composition, e.g. more than 150 g starch material per kg dried composition, such as more than 200 g starch material per kg dried composition, e.g. more than 250 g starch material per kg dried composition, such as more than 300 g starch material per kg dried composition.

It may be preferred that the anti-parasitic composition according to the present invention may be a dried composition. Preferably having a moisture content of 15% (w/w) or less, such as less than 12.5% (w/w), e.g. less than 10% (w/w), such as less than 7.5% (w/w), e.g. less than 5% (w/w).

The at least one plant material and/or the at least one seaweed material may have an average maximum diameter of 5 cm, such as an average maximum diameter of 4 cm such as an average maximum diameter of 3 cm, such as an average maximum diameter of 2 cm, such as an average maximum diameter of 1 cm, such as an average diameter in the range 25 μm to 5 cm, such as 0.1 mm to 5 cm, such as an average diameter in the range of 0.5 mm to 5 cm, such as an average diameter in the range 0.5 mm to 2 cm.

In an embodiment of the present invention the anti-parasite composition may be a fraction obtained from the at least one seaweed material (preferably at least one fermented seaweed material), at least one plant material (preferably at least one fermented plant material) or a combination of at least one seaweed material (preferably at least one fermented seaweed material) and at least one plant material (preferably at least one fermented plant material).

In the present context the term “fraction” relates to a composition obtained from the at least one seaweed material, and/or the at least one plant material. Preferably. the fraction according to the present invention does not comprises one or more of a fibrous material; a protein material; an enzyme material or microorganisms from the at least one seaweed material, and/or the at least one plant material. Preferably, the fraction according to the present invention does not comprises at least the fibrous material from the at least one seaweed material, and/or the at least one plant material.

In a preferred embodiment the fraction according to the present invention may be a non-polar fraction. Preferably, obtained from the at least one seaweed material, and/or the at least one plant material.

In a preferred embodiment of the present invention the non-polar fraction may be a fraction comprising one or more fatty acid compounds. Preferably, the non-polar fraction may comprise one or more modified fatty acid compounds.

The non-polar fraction may comprise several different structures of fatty acid compounds, preferably, modified fatty acid compounds.

In an embodiment of the present invention the non-polar fraction may comprise one or more fatty acid compounds. Preferably, the one or more fatty acid compounds may be at least one C18-fatty acid compound or at least one modified C18-fatty acid compound. In a further embodiment of the present invention the at least one fatty acid compound and/or the at least one C18 compound is at least one linolenic acid compound, a modified linolenic acid compound or a derivative thereof; preferably, the at least one C18 compound is at least one modified linolenic acid compound or a derivative thereof.

In an embodiment of the present invention the anti-parasite composition may be formulated into a pellet, a mash, a tablet, a pill, a sachet, a crème, an ointment, an extruded wet cake or an oil. Preferably, the anti-parasitic composition may be provided as a dried powder, a sachet, an extruded wet cake, a mash a pellet for the oral administration. Preferably, the anti-parasitic composition may be provided as a pill or a tablet, or a suspended slurry for oral or rectal administration. Preferably, the anti-parasitic composition is provided as a crème, an ointment, or an oil for topical administration.

The anti-parasitic composition according to the present invention maybe suitable for treating, alleviating and/or preventing growth of at least one parasite.

In an embodiment of the present invention the anti-parasitic composition according to the present invention maybe suitable for treating, alleviating and/or preventing growth of at least one parasite in a human or in an animal, such as fish (in particular salmon species), pigs, broiler chickens, roaster chickens, lying hens, turkey; ducks, geese, quails, cows, dogs, sheep, goats, cats and horses.

In the context of the present invention, the term “treatment” relates to the use of the anti-parasitic composition according to the present invention, in an attempt to cure or mitigate a disease, a condition, or an injury in a human or an animal.

The term “alleviation” used in the present invention relates to the action of the anti-parasitic composition according to the present invention, to make a disease, a condition or an injury less intense and/or reduce symptoms in a human or an animal.

In the context of the present invention the term “prophylaxis” relates to the use of the anti-parasitic composition according to the present invention, in an attempt to prevent a disease, a condition or an injury in a human or an animal, and/or for the protective treatment of a human or an animal.

In the present context the animal relates to fish (in particular salmon species), pigs, broiler chickens, roaster chickens, lying hens, turkey; ducks, geese, quails, cows, dogs, sheep, goats, cats and horses.

The anti-parasitic composition according to the present invention may be used directly or as part of or as an ingredient of another product.

A preferred embodiment of the present invention relates to a feed product comprising the anti-parasitic composition according to the present invention. Preferably the feed product is a fish feed product, in particular a fish feed product for salmonids.

In the event the anti-parasitic composition according to the present invention may be used as part of or as an ingredient of another product the feed product may comprise a content of the anti-parasitic composition in the range of 0.5-25% (w/w), such as in the range of 1-20% (w/w), e.g. in the range of 2-18% (w/w), such as in the range of 3-16% (w/w), e.g. in the range of 5-15% (w/w), such as in the range of 7-12% (w/w), e.g. in the range of 8-10% (w/w).

In a preferred embodiment of the present invention the anti-parasitic composition may provide inhibiting, reducing and/or suppressing effect on the growth of at least one parasite in a food product and/or in a feed product as well as inhibiting, reducing and/or suppressing effect (or treating, alleviating and/or preventing growth) of at least one parasite in a human or animal consuming or using the anti-parasitic composition.

When the anti-parasitic composition comprises a fermented seaweed material; a fermented plant material, or the combination of a fermented seaweed material and a fermented plant material, the fermentation process may be performed by adding an inoculum comprising the fermenting microorganisms according to the present invention. Preferably the fermentation process may be an anaerobic fermentation process. Even more preferably the fermentation process for providing an anti-parasitic composition comprising fermented plant material and/or fermented seaweed according to the present invention may be an anaerobic homolactic fermentation process.

The fermented seaweed material and/or the fermented plant material, may preferably not be subjected to sterilisation in order to maintain the native metabolic nature of the materials.

The inoculum may be provided with a concentration and/or in an amount of lactic acid bacteria in the inoculum sufficient to outgrow any bacteria, yeast or moulds present in the product seaweed material and/or the plant material.

Specific conditions for providing and fermenting the fermented seaweed material; the fermented plant material, or the combination of fermented seaweed material and fermented plant material, may be as described in WO 2008/006382; WO 2014/206419; or PCT/EP2016/076952. WO 2008/006382; WO 2014/206419; or PCT/EP2016/076952 are hereby incorporated by reference.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

Example 1: Sensitivity test of salmon lice (Lepeophtheirus salmonis) with fermented rapeseed and seaweed. Lethal dose 50 and 95% determinations (LD50 and LD95).

### Method

Salmon lice parasite the skin, gills and blood of salmon creating major zoonotic problems for the salmon industry. One of the big challenges are the increasing resistance in salmon louse to the available agents used for treatment. The industry has also increasing interest in replacing chemical treatments with greener solutions.

This parasite belonging to the subclass copepoda has several life stages. The first life stage Nauplius is as free living plankton, and the remaining stages as parasites of salmon. The following experiment was carried out to measure the extent of sensitivity of live louse copepods obtained directly from commercial reared salmons, in terms of LD50 and LD95.
1. 20 adult and 20 younger (chalimus) salmon louse copepods were placed in glass beakers with sterile sea water, in triplicate.
2. 10 g of fermented rapeseed and seaweed (mixture of Saccharina latissimi and Ascophyllum nodosum) were extracted in 100 mL of cold methanol using Ultra-turrax for vigorous mixing. The supernatant was transferred to a glass vial and dried under nitrogen flow. Prior to the experiment, the pellet was reconstituted 100 mL of DMSO-water mixture (1:1 vol:vol) giving a final concentration of 150 mg/ml.
3. The suspension containing the fermented extract was titrated in different doses into the sterile sea water (2, 5, 10, 20% vol:vol).
4. The beakers were cover and incubated at room temperature with direct natural light for 24 h.
5. After incubation, mortality of copepods was assessed by their motility under a micro-stereoscope visualization. LD50 and LD95 were determined, where (−) indicates an unsuccessful lethal dosage and (+) indicates a successful lethal dosage (see table 1).

### Results

TABLE 1
Toxicity level of fermented inducing mortality in
the salmon lice (adult and young) population.

| Titration of | Final | Mortality of | | Mortality of | |
|---|---|---|---|---|---|
| ferment | concentration | adult salmon | | younger chalimus | |
| extraction | (mg/mL sea | lice | | salmon lice | |
| (%) | water) | LD₅₀ | LD₉₅ | LD50 | LD95 |
| Fermented rapeseed-seaweed | | | | | |
| 2 | 3 | − | − | − | − |
| 5 | 7.5 | − | − | − | − |
| 10 | 14 | + | − | + | − |
| 20 | 28 | + | − | + | − |
| Fermented seaweed | | | | | |
| 2 | 3 | + | − | + | − |
| 5 | 7.5 | + | − | + | − |
| 10 | 14 | + | + | + | + |
| 20 | 28 | + | + | + | + |

In Vitro Experimental Results Used for their Application in the Field

The results from the in vitro experiment, were used as reference for a controlled challenge-trial. Adult salmon fishes in closed tanks were parasitized with salmon lice copepods. The trial consisted of 4 repetitions of positive and negative control groups, and 4 repetitions of 10 and 20% fermented rapeseed-seaweed (mixture of Saccharina latissimi and Ascophyllum nodosum) and fermented seaweed (mixture of Saccharina latissimi and Ascophyllum nodosum) dietary inclusion, dry basis (Table 2). All treatment groups had the same commercial basal diet. With exception of the negative control, the treatment groups differentiated by the addition of either a commercial salmon lice chemical treatment or fermented rapeseed seaweed feed additives. Two different treatments were experimented separately as positive controls (Table 2). The salmons were fed the experimental treatments for 15 days, in the case of Salmonsan® Vet, the product was applied following manufacturer instructions during that period.

After this period, the fishes were slaughtered, and the number of salmon lice attached to the salmons was counted per tank level. In addition, measurements on body size and weight were also registered in all treatment groups at tank level. Results are displayed in table 3.

TABLE 2
Overview of dietary treatments, total no. of repetitions
(tanks) and total no. of salmons per TG.

| | | Number of | Number of |
|---|---|---|---|
| Treatment | | repetitions | salmons per |
| group (TG) | Description | (tank) | repetition/tank |
| TG1 | Basal commercial diet | 4 | 80 |
| | (negative control) | | |
| TG2 | Basal commercial diet + | 4 | 80 |
| | Salmosan ® vet | | |
| | (positive control) | | |
| TG3 | Basal commercial diet + | 4 | 80 |
| | Releeze ® vet | | |
| | (positive control) | | |
| TG4 | Basal commercial diet + | 4 | 80 |
| | 10% fermented rapeseed- | | |
| | seaweed | | |
| TG5 | Basal commercial diet + | 4 | 80 |
| | 20% fermented rapeseed- | | |
| | seaweed | | |
| TG6 | Basal commercial diet + | 4 | 80 |
| | 10% fermented seaweed | | |
| TG7 | Basal commercial diet + | 4 | 80 |
| | 20% fermented seaweed | | |

TABLE 3
Overview of number of salmon lice attached to fish (all
stages), body weight and size as per tank level per
TG, after 15 days of treatment supplementation.

| | TG1 | TG2 | TG3 | TG4 | TG5 | TG6 | TG7 |
|---|---|---|---|---|---|---|---|
| Average number | 40 | 5 | 10 | 9 | 4 | 2 | 2 |
| of copepods | | | | | | | |
| attached/salmon | | | | | | | |
| Average weight | 20.1 | 26.5 | 25.4 | 23.4 | 27.5 | 28.0 | 28.5 |
| gain (kg) | | | | | | | |
| Average size | 71.2 | 76.4 | 75.5 | 77.3 | 77.3 | 77.2 | 76.8 |
| (cm) | | | | | | | |

### REFERENCES

WO 2008/006382
WO 2014/206419
PCT/EP2016/076952

1-15. (canceled)

16. A method for inhibiting, reducing and/or suppressing growth of at least one parasite by administering an anti-parasitic composition comprising at least one seaweed material or a combination of at least one seaweed material and at least one plant material, wherein the at least one seaweed material is a fermented seaweed material and/or wherein the at least one plant material is a fermented plant material and wherein the composition comprises more than 5 g fibrous material per kg dried anti-parasitic composition.

17. The method according to claim 16, wherein the anti-parasitic composition comprise at least one lactic acid bacterial strain.

18. The method according to claim 16, wherein the at least one plant material and/or the at least one seaweed material has an average maximum diameter of 5 cm, such as an average maximum diameter in the range 25 μm to 5 cm.

19. The method according to claim 16, wherein the composition comprises a pH-value below pH 6.5, such as below pH 6.0, e.g. below pH 5.5, such as below pH 5.0, e.g. in the range of pH 3.0-6.5, such as in the range of pH 3.0-6.0, e.g. in the range of pH 3.1-5.5, such as in the range of pH 5.0-3.2, such as in the range 3.3-4-2, such as 3.4-4.0, such as 3.5-3.8, such as 3.7-4.2, such as 3.7-4.0, or such as 3.8-4.2.

20. The method according to claim 16, wherein the composition comprises a lactic acid concentration of at least 50 mM, such as at least 100 mM, such as 100-1000 mM, such as 100-500 mM, such as 100-300 mM, such as 100-200 mM, such as 150-500 mM, such as 200-500 mM or such as 300-500, mM lactic acid.

21. The method according to claim 16, wherein the seaweed material is a unicellular alga or a multicellular macroalgae, preferably, the multicellular macroalgae may be selected from brown macroalgae, red macroalgae, and/or green macroalgae, preferably the brown macroalgae may be selected from one or more of kelps, Saccharina latissimi (Laminaria saccharina), Laminaria digitate, Ascophyllum nodosum, Laminaria hyperborean, or a mixture hereof.

22. The method according to claim 16, wherein the plant material may be selected from at least one proteinaceous plant material, preferably, the proteinaceous plant material is selected from eudicot plants, angiosperm plants, and/or rosid plants, preferably, the proteinaceous plant material is selected from Brassicale plants, preferably, the Brassicale plants is selected from the Brassicaceae family or the Cruciferae family.

23. The method according to claim 16, wherein the parasite is an ectoparasites or an endoparasite.

24. The method according to claim 16, wherein the anti-parasitic composition may be provided as a dried powder, a sachet, an extruded wet cake, a mash a pellet for the oral administration.

25. A method of administering an anti-parasitic composition according to claim 16, for treating, alleviating and/or preventing growth of at least one parasite.

26. A feed product comprising an anti-parasitic composition comprising at least one seaweed material or a combination of at least one seaweed material and at least one plant material, wherein the at least one seaweed material is a fermented seaweed material and/or wherein the at least one plant material is a fermented plant material and wherein the composition comprises more than 5 g fibrous material per kg dried anti-parasitic composition.

27. The feed product according to claim 26, wherein the feed product is a fish feed product, in particular a fish feed product for salmonids.

28. The feed product according to claim 26, wherein the feed product comprises a content of the anti-parasitic composition in the range of 0.5-25% (w/w), such as in the range of 1-20% (w/w), e.g. in the range of 2-18% (w/w), such as in the range of 3-16% (w/w), e.g. in the range of 5-15% (w/w), such as in the range of 7-12% (w/w), e.g. in the range of 8-10% (w/w).

29. A method for treatment, prophylaxis and alleviation of a parasitic infection by administering an anti-parasitic composition comprising at least one seaweed material or a combination of at least one seaweed material and at least one plant material, wherein the at least one seaweed material is a fermented seaweed material and/or wherein the at least one plant material is a fermented plant material and wherein the composition comprises more than 5 g fibrous material per kg dried anti-parasitic composition.

30. A method for inhibiting, reducing and/or suppressing growth of at least one parasite in a food product and/or in a feed product by adding an anti-parasitic composition to the food product and/or the feed product, wherein the anti-parasitic composition comprising at least one seaweed material or a combination of at least one seaweed material and at least one plant material, wherein the at least one seaweed material is a fermented seaweed material and/or wherein the at least one plant material is a fermented plant material and wherein the composition comprises more than 5 g fibrous material per kg dried anti-parasitic composition.
